(19) United States
(12) Patent Application Publication
(10) Pub. No.: US 2026/0301224 A1
(43) Pub. Date: Oct. 1, 2026

(54) AUGMENTING 3D PATIENT SCANS WITH CAD MODEL OF IMPLANTED OBJECT

(71) Applicant: GLOBUS MEDICAL, INC., AUDUBON, PA (US)

(72) Inventors: Sanjay M. Joshi, Andover, MA (US); Norbert Johnson, North Andover, MA (US); Samuel Paster, Jamaica Plain, MA (US)

(21) Appl. No.: 19/690,724

(22) Filed: May 28, 2026

Related U.S. Application Data
(63) Continuation of application No. 18/531,766, filed on Dec. 7, 2023, now Pat. No. 12,657,769.

(51) Int. Cl. G06T 7/73; G06T 7/33; G06T 7/55; G06T 7/60; G06T 19/00

(52) U.S. Cl. CPC G06T 7/75; G06T 7/344; G06T 7/55; G06T 7/60; G06T 19/00; G06T 2207/30004; G06T 2210/41

(57) ABSTRACT

A computer implemented method includes obtaining a plurality of 2D images of an object implanted in a patient and obtaining a computer-aided design (CAD) model corresponding to the object. The method further includes augmenting a 3D scan of the patient to provide a representation of the CAD model with a pose in a reference system of the 3D scan determined based on pose of the object in the 2D images.

### CROSS-REFERENCE TO RELATED APPLICATIONS

This application is a continuation application of U.S. patent application Ser. No. 18/531,766, filed on Dec. 7, 2023 and published as U.S. 2025-0191225, the entirety of which is incorporated herein by reference.

### FIELD

The present disclosure relates to medical imaging, and in particular, to processing 3D scans of patients, including for computer-aided surgical planning and/or intraoperative navigation using a 3D scan of a patient.

### BACKGROUND

Computer-aided surgeries have been using 3D medical imaging scans (“3D scans”) of patients for planning and intraoperative navigation. High-quality 3D scans usually require large imaging equipment, such as Computerized Tomography (CT) or Magnetic Resonance Imaging (MRI) equipment, typically situated in a radiology department but not available in operating rooms. The 3D scans can be registered to 2D intraoperative images obtained with readily available x-ray equipment in the operating room, such as by C-Arms. The poses of 2D x-ray images are tracked with a navigation camera, yielding their pose in camera space. Using a 2D-3D registration transform, intraoperative surgical navigation on high-quality 3D images can be provided.

Visualization of navigated instruments and patient anatomy in 3D relies on correct representation of patient anatomy and implants in 3D scans. When an object, such as a metal screw, is implanted in a patient's body after the 3D scan is taken, that object is not visible in the 3D scan. Unfortunately, a surgeon may then plan implantation of another object or trajectory that may come too close or collide with the earlier implanted object.

If an intraoperative 3D imaging system is available, such as Excelsius3D by Globus Medical Inc., it is possible to take a new 3D scan that shows the object. However, the potential benefit of this additional 3D scan may be outweighed by the desire to avoid additional radiation exposure to the patient.

### SUMMARY

Some embodiments of the present disclosure are directed to a computer implemented method that includes obtaining a plurality of two-dimensional (2D) images of an object implanted in a patient, and obtaining a computer-aided design (CAD) model corresponding to the object. The method further includes augmenting a three-dimensional (3D) scan of the patient to provide a representation of the CAD model with a pose in a reference system of the 3D scan determined based on pose of the object in the 2D images.

Some other related embodiments are directed to a computer processing platform that includes at least one network interface, at least one processor connected to communicate through the at least one network interface, and at least memory storing instructions executable by the at least one processor to perform operations. The operations include obtaining a plurality of 2D images of an object implanted in a patient, and obtain a computer-aided design (CAD) model corresponding to the object. The operations further include augmenting a 3D scan of the patient to provide a representation of the CAD model with a pose in a reference system of the 3D scan determined based on pose of the object in the 2D images.

These and other methods, computer processing platforms, and computer program products according to embodiments will be or become apparent to one with skill in the art upon review of the following drawings and detailed description. It is intended that all such methods, computer processing platforms, and computer program products be included within this description, be within the scope of the present disclosure, and be protected by the accompanying claims. Moreover, it is intended that all embodiments disclosed herein can be implemented separately or combined in any way and/or combination

### BRIEF DESCRIPTION OF THE FIGURES

Aspects of the present disclosure are illustrated by way of example and are not limited by the accompanying drawings. In the drawings:

FIG. 1 illustrates two fluoroscopy images rotationally offset by 90 degrees relative to an implanted object which is stationary with respect to a DRB;

FIG. 2 illustrates an operational flowchart of an algorithm that estimates pose of a computer-aided design (CAD) model relative to an implanted object imagined in two rotationally offset 2D images in accordance with some embodiments;

FIG. 3 illustrates two 2D x-ray images of a surgical instrument (object) at rotationally offset poses of an x-ray detector (imager), which can be processed to estimate pose of a CAD model of the imaged surgical instrument in accordance with some embodiments;

FIG. 4 illustrates operations for reconstruction of one plane of a 3D volume in accordance with some embodiments;

FIG. 5 illustrates impact of image boundary digitization for different cross-sectional areas of an object in accordance with some embodiments;

FIG. 6 illustrates the impact of an object not being in an optimal pose for two x-ray images to be processed for planar reconstruction in accordance with some embodiments;

FIG. 7 illustrates operations for iterative refinement of boundary digitization using three images in accordance with some embodiments;

FIG. 8 illustrates operations for boundary digitization of an object having a concave surface, in accordance with some embodiments;

FIG. 9 illustrates operations using four image orientations to reconstruct a 2×2 cross-sectional area of an object in accordance with some embodiments; and

FIG. 10 illustrates another operational flowchart of an algorithm that augments a 3D scan of a patient to represent a CAD model corresponding to an object implanted in the patient, and which is used to perform pre-operative computer-aided planning and intraoperative computer-aided navigation in accordance with some embodiments.

### DETAILED DESCRIPTION

Inventive concepts will now be described more fully hereinafter with reference to the accompanying drawings, in which examples of embodiments of inventive concepts are shown. Inventive concepts may, however, be embodied in many different forms and should not be construed as limited to the embodiments set forth herein. Rather, these embodiments are provided so that this disclosure will be thorough and complete, and will fully convey the scope of various present inventive concepts to those skilled in the art. It should also be noted that these embodiments are not mutually exclusive. Components from one embodiment may be tacitly assumed to be present or used in another embodiment.

As explained above, when an object is implanted into a patient's body after the 3D scan is taken, that object is not visible in the 3D scan. A surgeon may therefore unknowingly plan another object or trajectory that may come too close or collide with the earlier implanted object.

Various embodiments of the present disclosure are directed to using a 2D imaging modality to image an implanted object and then register a computer-aided design (CAD) model of the imaged object to a 3D scan of the patient using a 3D imaging modality. The augmented 3D scan may then be used for pre-operative computer-aided planning and intraoperative computer-aided navigation.

The 3D imaging modality may include, but is not limited to, a computerized tomography (CT) scan (e.g., via a C-arm imaging system or O-arm imaging system), a magnetic resonance imaging (MRI) scan (e.g., via a MRI system), Cone Beam Computerized Tomography (CBCT), Micro Computerized Tomography (MCT), 3D ultrasound, etc. The 2D imaging modality may include, but is not limited to, x-ray imaging, fluoroscopy imaging, ultrasound, etc. Although some embodiments are described in the context of fluoroscopy images or other x-ray images, the images processed by these and other embodiments are not limited to any particular imaging modality and images from different imaging modalities may be combined using these embodiments to provide digital reconstruction of the imaged object.

Fluoroscopy imaging systems optimize kV-mA settings to minimize radiation dose while ensuring patient tissue contrast. At these settings, implanted objects such as metal screws and interbodies are radiographically opaque and show up as almost complete black. Some operational embodiments of the present disclosure use this property to treat the fluoroscopy image as a ‘binary’ image, e.g., black where metal implants are present and white where they are not.

The term “object” can include, but is not limited to, radiographically opaque objects such as screws and interbodies, radiographically ‘dark’ contrast agents flowing through anatomy of known shape, such as cylindrical blood vessels, etc.

The pose of the object relative to the fluoroscopy images is used as described below. The pose can be estimated in the following two scenarios.

In the first scenario, the operations can assume that the pose of the object does not change with respect to the tracking camera which is tracking pose of the 2D image device. This is possible if the camera is secured in one pose and the patient stays immobilized while the images are acquired with angular offsets relative to the patient. These operations thereby only need to use a few fluoroscopy images, e.g., two or more images.

In the second scenario, the tracking camera needs to be moved between Fluoroscopy images to ensure visibility of the tracking array of the Fluoroscope to the tracking camera. In this scenario, a reference element, such as Dynamic Reference Base (DRB), is securely attached to the patient and tracked by the tracking camera to ensure that the relative pose of the object in patient with respect to the DRB does not change. In this scenario, all the computations can be performed in the DRB reference space because the tracking camera can track the DRB, and the corresponding poses in the camera space can be readily calculated.

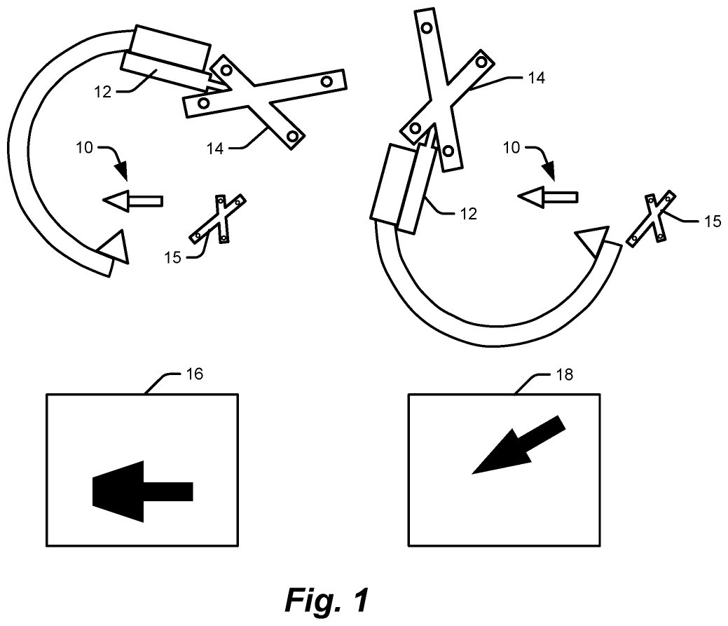

FIG. 1 illustrates two fluoroscopy images 16 and 18 that are rotationally offset by 90 degrees relative to an implanted object 10 which is stationary with respect to a DRB 15.

Referring to FIG. 1, an imager 12, e.g., detector, of a fluoroscopy system outputs image 16 of object 10. Imager 12 is rotated about 90 degrees and then outputs image 18 of object 10. Relative pose of the imager 12 to the DRB 15 can be tracked by a tracking camera which tracks pose of a tracking fixture 14 (e.g., reference array) attached to the imager 12 and further tracks pose of the DRB 15. Alternatively or additionally, the imaging device may directly track and record the pose of the imager 12 when capturing images.

Operational approaches are now explained which can be performed to identify and register a CAD model of an imaged object, e.g., object 10, to a 3D scan of the patient, which may be performed alternatively or in full or partial combination.

### First Approach—Known Object Geometry:

A first operational approach is adapted to be used when the geometry of the object is known to a computer processing platform (also “computer platform”). The computer platform may have access to a database of implantable objects, which defines for each object a CAD model that defines shape and physical dimensions. Alternatively, the computer platform may receive information defining the CAD model from a user or an electronic component of the system. For example, when the computer platform has access to the CAD model and dimensions of the implant (e.g., CREO screw, 40 mm long, 5.5 mm diameter), the problem to be solved reduces to identifying the pose of the CAD model that correspond to the two fluoroscopy images. The pose can be directly estimated by locating the defined features of the CAD model projected into both images.

In accordance with one embodiment, a computer platform includes at least one network interface, at least one processor connected to communicate through the at least one network interface, and at least memory storing instructions executable by the at least one processor to perform operations. The operations obtain a plurality of 2D images of an object implanted in a patient, and obtain a CAD model corresponding to the object. The operations augment a 3D scan of the patient to provide a representation of the CAD model with a pose in a reference system of the 3D scan determined based on pose of the object in the 2D images.

The operation to augment the 3D scan of the patient can include rendering a graphical representation of the object based on the CAD model, and overlay the graphical representation of the object with the pose in the reference system of the 3D scan. The CAD model may be obtained based on selecting the CAD model from a set of CAD models residing in a memory based on information identify the CAD model as corresponding to the object.

The CAD model may define shape and dimensions of the object, and the operation to augment the 3D scan of the patient may provide the representation of the CAD model scaled based on the dimensions of the object defined by the CAD model and posed in the reference system of the 3D scan based on the pose of the object in the 2D images. The operations can further include to project features of the shape defined by the CAD model into the 2D images, and determine the pose of the CAD model in the reference system of the 3D scan based on correlating features of the object in the 2D images to the projected features of the shape defined by the CAD model.

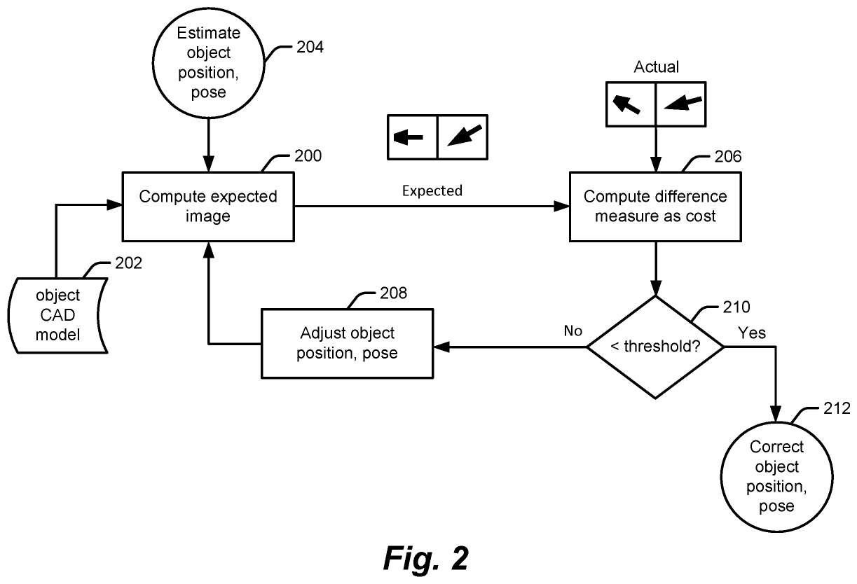

FIG. 2 illustrates an operational flowchart of an algorithm that estimates the pose of a CAD model relative to an implanted object captured in rotationally offset 2D images in accordance with this first operational embodiment.

Referring to FIG. 2, a computing platform performs operations 200 to compute expected images of an object based on an object CAD model 202, e.g., from a database of implantable objects, and based on estimated 204 object poses (e.g., position and angular orientation) which can be determined based on tracked poses of the imaging device when actual images of the object are acquired. The expected images of the object are compared to actual images of the object output by the imaging device to compute 206 a difference measure as a cost. The comparison of the expected images to the actual images of the object can be more generally perform as a correlation operation that measures similarity between how the CAD model based object is expected to appear in a set of images and how the object actually appears in the actual images.

When the cost is determined 210 to not satisfy a rule (e.g., exceeds a threshold), operations adjust 208 the estimate of the object pose and another cycle of operations are performed to compute 200 another set of expected images of the object and compute 206 the difference between the expected and actual images of the object until the cost is determined 210 to satisfy the threshold.

In contrast, when the cost is determined 210 to satisfy the rule (e.g., is less than the threshold), the object CAD model and pose are output 212. The object CAD model and pose can be used to provide (e.g., overlay or otherwise insert) a CAD model based rendering of the object in one or more 2D images and/or in a 3D scan of the patient. The object CAD model and pose may be used to generate a computer generated representation of the object with a pose that is registered to one or more patient images and/or 3D scans used for pre-operative computer-aided planning and/or intraoperative computer-aided navigation.

In accordance with a further embodiment, corresponding operations to project the features of the shape defined by the CAD model into the 2D images and to determine the pose of the CAD model in the reference system of the 3D scan, can include to estimate pose of the object in the 2D images, compute expected object images based on the features having the shape defined by the CAD model projected into the 2D images using the estimated pose of the object in the 2D images. The operations further include to compare the expected object images to the 2D images of the object to compute a difference measure, and determine whether the difference measure satisfies a rule. When the difference measure satisfy the rule, the operations determine a pose of the CAD model in a 3D space based on the estimating pose of the object in the 2D images, and determine the pose of the CAD model in the reference system of the 3D scan based on the pose of the CAD model in the 3D space.

### Second Approach—Known Approximate Object Shape:

A second operational approach is adapted to be used when an approximate (partial) shape of the object is known to the computer platform. For example, when the object has a known shape of a cylinder, rectangular block, screw, etc., the measurements of the shape, such as length and diameter of that known shape can be estimated from the actual images (e.g., Fluoro images) using image processing techniques that detect surface lines (e.g., straight lines, etc.). The operations can assume that a point in one of the images corresponds to a line in another one of the images, when the object is imaged from orthogonal directions. In this manner, by identifying one point along this line the operations can determine therefrom the location of the point in a 3D space. The approximate shape and determined location of the object in the 3D space can be used to identify the corresponding CAD model of the object. The pose of the CAD model can then be calculated based on the operations discussed above with regard to FIG. 2 using the now-identified CAD model.

A corresponding operational embodiment when the CAD model defines shape of the object (e.g., but does not define dimensions) can include to correlate features of the shape of the object defined by the CAD model to corresponding features of the object in the 2D images, estimate dimensions of the corresponding features of the object in the 2D images, and estimate dimensions of the shape of the object defined by the CAD model based on the estimated dimensions of the corresponding features of the object in the 2D images.

### Third Approach—Unknown Object Shape:

A third operational approach is adapted to be used when, for example, insufficient information is known about the object shape to enable use of the first approach or second approach described above, such as when nothing about the object shape is known. The third operational approach enables reconstruction of the object shape in 3D using a plurality of fluoroscopy images. The number of images that are used may be 2, 3, or other relatively small number that is much less than, for example, the 360 images (or more than 180) projection images used in conventional CT scans.

Accurate reconstruction of 3D data for an object is generally not possible using only two x-ray images. The back-propagation algorithm used in CT reconstruction typically uses tens of 2D x-ray images to reconstruct 3D voxel data (e.g., for a typical CT scanner, more than 180 fan beam angle images). The 2D x-ray images represent a “sum” of absorption of x-rays in voxels along their paths, hence this many image samples are needed for a reliable reconstruction. In contrast, binary voxel data enables a different operational approach that enables reconstruction of 3D data for an object with fewer x-ray images as described below.

In binary 2D x-ray images, pixels are bright (Binary 1) where the x-rays (“rays”) travelling from an emitter to a detector (imager) go through all voxel containing radio-translucent material. All other dark (Binary 0) pixels are the result of blockage of rays by one or more voxels containing radio-opaque material, i.e., of the object.

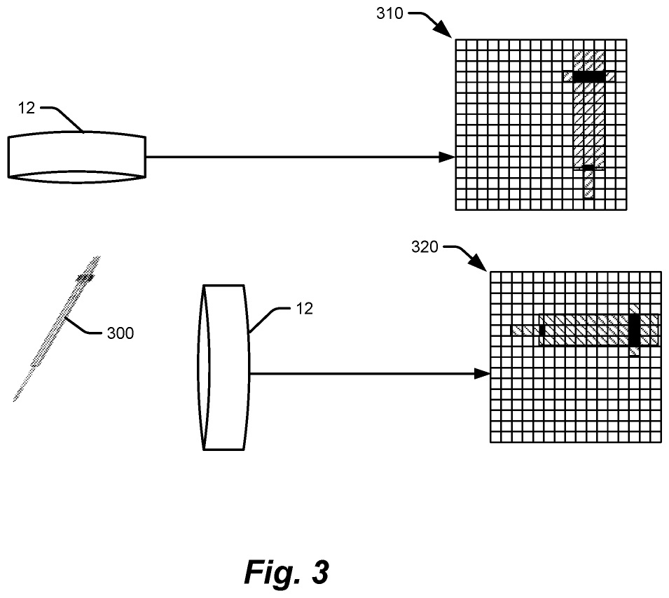

FIG. 3 illustrates two 2D x-ray images 310 and 320 of a surgical instrument (object) 300 at rotationally offset poses of an x-ray detector (imager) 12, which can be processed to estimate pose of a CAD model of the imaged surgical instrument in accordance with some embodiments.

Referring to FIG. 3, image 310 shows how the surgical instrument 300 can appear when detector 12 is posed above the surgical instrument to image x-rays from the emitter oppositely posed below the surgical instrument 300. The other image 320 shows how the surgical instrument 300 can appear when detector 12 is posed right of the surgical instrument 300 to image x-rays from the emitter oppositely posed left of the surgical instrument 300.

Another operational embodiment processes two or more x-ray images of binary 3D data which have known poses of the emitter and detector (imager). The poses of the emitter and/or detector can be electronically sensed by the imaging device when the images are acquired and can be indicated with the images, e.g., as metadata provided with the images. Alternatively, when the imaging device itself does not provide the emitter and/or detector poses, a tracking fixture (reference array) can be attached to the detector and tracked in 3D space by a tracking camera, to provide an estimate of detector and emitter poses (e.g., in accordance with an intraoperative Fluoroscopy workflow of ExcelsiusGPS by Globus Medical). For each image (scan), operations create a back-propagation pyramid with the emitter at the apex and the detector at the base, and configured so that the number of rays are equal to the number of pixels in the 2D image. When these pyramids (e.g., two back-propagation pyramids) are superimposed (e.g., through a binary OR operation), the resulting output can correspond to a 3D frustrum, with voxel values of 1 and 0. The output can be padded with zeros to create a rectangular 3D volume.

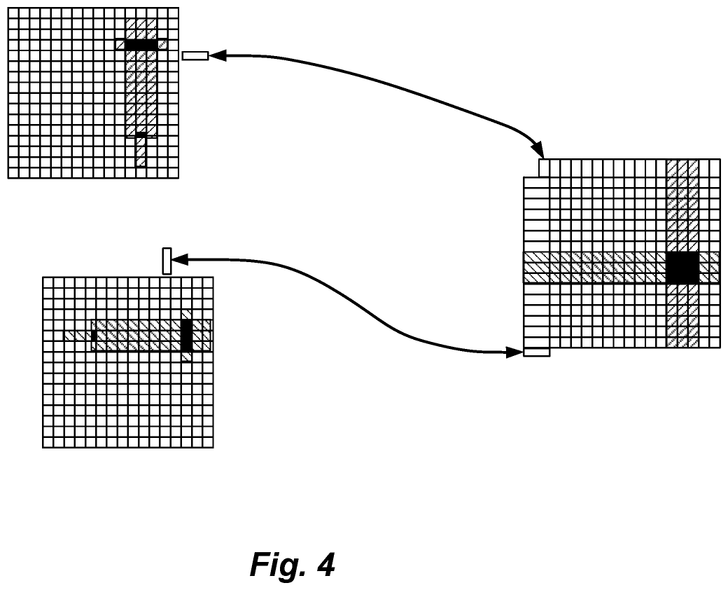

FIG. 4 illustrates corresponding operations for reconstruction of one plane of a 3D volume in accordance with some embodiments. The operations assume two x-ray images with normals orthogonal to each other and approximate each voxel to a cube. One row and one column from each x-ray image is processed with a binary OR operation to create one plane. The lighter gray areas represent the reconstructed path of rays. The black area represents where the cross-hatched paths intersect. Multiple such planes, parallel to each other, create the 3D volume.

The planar reconstruction may be most accurate, or may only be correct, if the actual (physical) object's cross-section in this plane has a substantially rectangular surface that is substantially aligned with the two x-ray images, or if the cross-section substantially fits within one pixel. For an actual (physical) object having a larger cross-section, the approximation into pixels can be provided a result of “boundary digitization.” For an actual (physical) object with a relatively small cross-sectional area (e.g., typically corresponding to less than 3 pixels per side), the digitized boundary of the actual surface can be a sufficiently accurate representation. For larger objects, however, the difference could be significant.

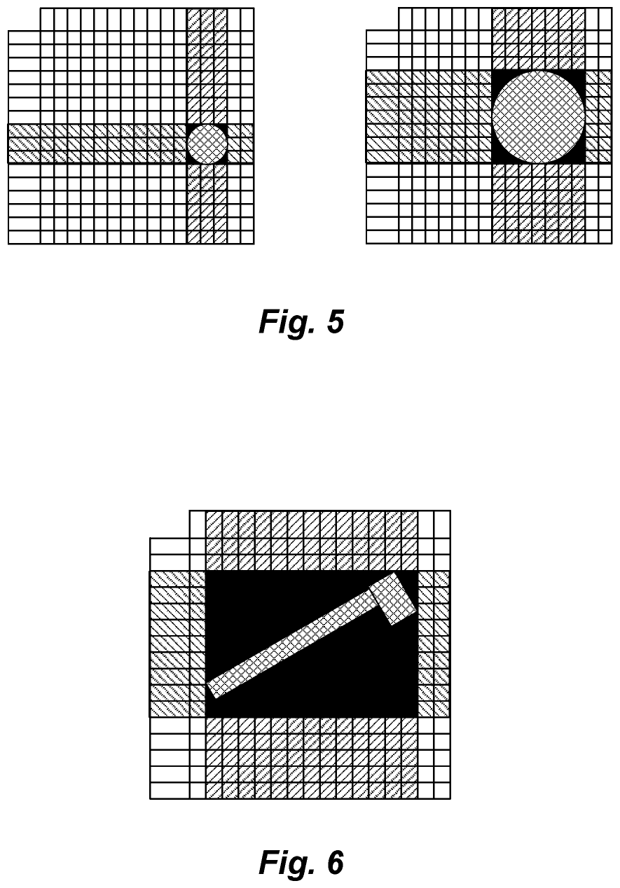

FIG. 5 illustrates the impact of image boundary digitization for different cross-sectional areas of an object in accordance with some embodiments. Referring to FIG. 5, an actual (physical) object has a circular cross-sectional area which is depicted as a circle enclosing cross-hatched lines. The digitized boundary of the object which is generated from the pair of images is depicted as the black square area which encloses the circular cross-sectional area. Accordingly, the curved object surface results in a level of error in the planar reconstruction operations.

The above scenarios can correspond to the case when the object that is imaged has a relatively small cross-sectional area in the x-ray images. When the object in FIG. 1 is rotated, however, the object's cross-sectional area can grow significantly, which can substantially affect the boundary digitization, such as shown in FIG. 6. FIG. 6 illustrates the impact of an object not being in an optimal pose for two x-ray images to be processed for planar reconstruction in accordance with some embodiments. Referring to FIG. 6, the actual cross-sectional area is illustrated by the hashed area while the digitized boundary of the object generated from the pair of images is depicted as the black rectangular area which encloses the actual cross-sectional area.

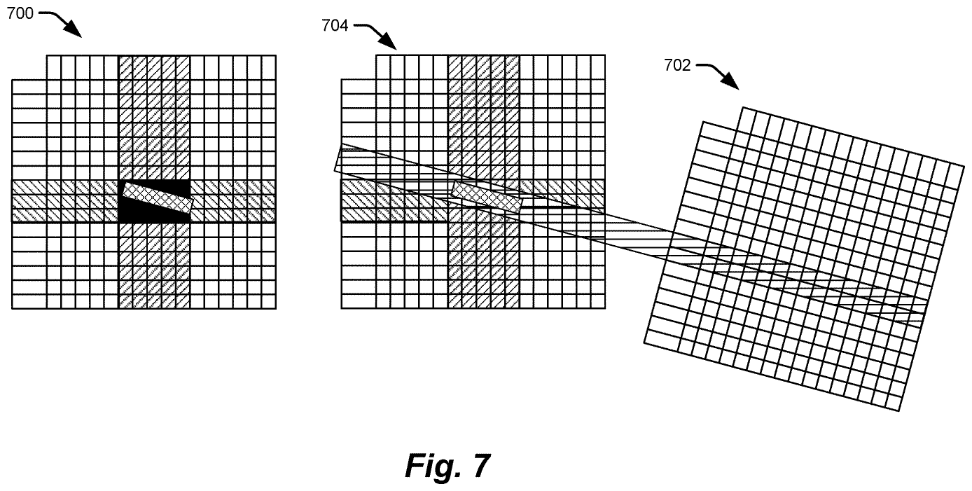

Accuracy of the planar reconstruction operations can be improved by using additional x-ray images of the object from offset poses. Corresponding pixels of the set of x-ray images are processed through the binary OR operation and iteratively refined. FIG. 7 illustrates operations for iterative refinement of boundary digitization using three x-ray images in accordance with some embodiments. Referring to FIG. 7, image 700 depicts the digitized boundary of the object's cross-sectional area initially generated from a pair of images. A subsequent combined image 704 is refined based a third image 702 which results in the more accurate digitized boundary of the object's cross-sectional area shown in the combined image 704.

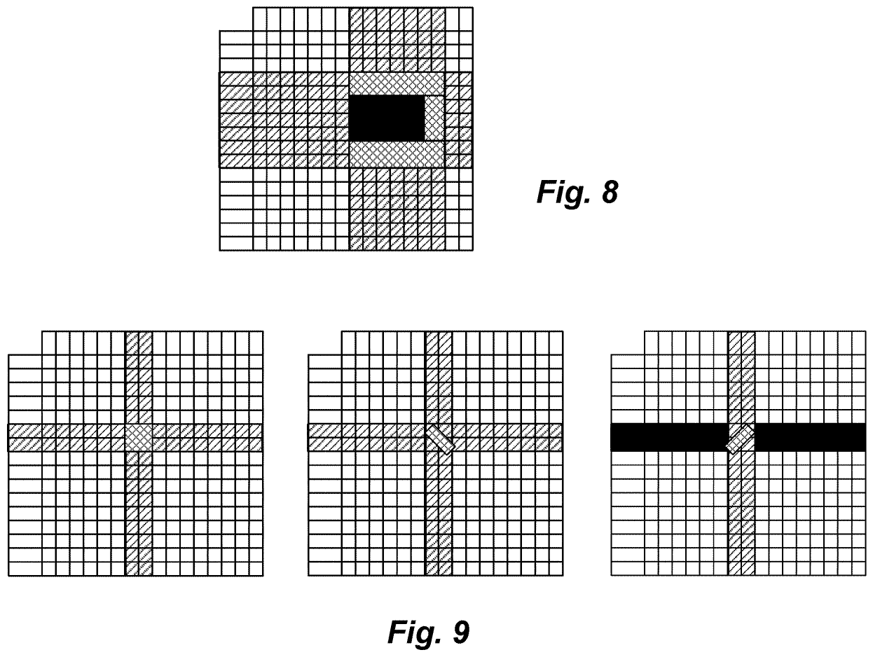

One potential limitation of this approach is that objects with only convex surface boundaries can be reconstructed with substantially improved results. FIG. 8 illustrates operations for boundary digitization of an object having a concave surface, in accordance with some embodiments. Referring to FIG. 8, it is observed that the object has a concave surface boundary, and where digitization of the concave surface boundary may not be able to be refined.

Some further embodiments are directed to operations which are directed to improving accuracy of digitization of objects.

In accordance with some embodiments, a mathematical model can be constructed on the minimum number of images needed to digitally reconstruct a cross-section of a number of pixels. In some example operations, the orientation of an image is defined as the slope of the line from the emitter to the center of the detector. The “horizontal” orientation has slope 0/1=0 and the ‘vertical’ orientation has slope 1/0=infinity. For a cross-section area of 1×1 pixel, only the 0/1 and 1/0 orientations are sufficient. A cross-section area of 2×2 pixels, however, would need two more orientations, at 1/1 and −1/1 orientation, as shown in FIG. 9.

FIG. 9 illustrates a scenario where an actual (physical) object should be imaged at four orientations to enable reconstruction of a 2×2 cross-section area with improved accuracy in accordance with one embodiment.

A corresponding operational embodiment can include to for each of the 2D images, to determine poses of an emitter and a detector of an imaging device corresponding to when the 2D image was generated by the imaging device, and to create a back-propagation pyramid estimation of the object in a 3D voxel array based on the object oriented in the 2D image corresponding to the emitter posed at an apex and the detector posed at the base of the back-propagation pyramid, and where the number of voxels in the 3D voxel array is based on a number of pixels in the 2D image. The operations can determine a 3D shape of the object in the 2D images based on superimposing the back-propagation pyramids. The superimposing of the back-propagation pyramids may include performing a binary OR operation on values at corresponding locations in the 3D voxel array of the back-propagation pyramids.

The number of image orientations needed to reliably reconstruct any convex shaped object cross-sectional area is shown in the table below.

| Area (number | Number of | |
|---|---|---|
| of pixels) | Orientations | Orientations (Slopes) |
| 1 × 1 | 2 | 0/1, 1/0 |
| 2 × 2 | 4 | 0/1, 1/1, -1/1, 1/0 |
| 3 × 3 | 8 | 0/1, 1/2, -1/2, 1/1, -1/1, |
| | | 2/1, -2/1, 1/0 |
| 4 × 4 | 14 | 0/1, 1/3, 2/3, |
| | | 1/2, -1/3, -2/3, -1/2, |
| | | 1/0, 3/1, 3/2, |
| | | 2/1, -3/1, -3/2, -2/1 |
| 5 × 5 | 22 | 0/1, 1/4, 3/4, 1/3, 2/3, |
| | | 1/2, -1/4, -3/4, -1/3, -2/3, |
| | | -1/2, 1/0, 4/1, 4/3, 3/1, |
| | | 3/2, 2/1, -4/1, -4/3, -3/1, |
| | | -3/2, -2/1 |

As the table shows, for an N×N cross-sectional area, the number of orientations necessary to reconstruct the area is less than N2 for N>1.

In an illustrative example, assume an x-ray scanner has a 12×12 inch panel, and the object to be imaged is positioned at ⅓rd distance away from detector panel, the imaged size is 9×9 inch, approximately 256 mm×256 mm. For a 512×512 image resolution, that translates to 0.5 mm/pixel resolution. Thus, objects with small cross-sectional areas, such as a surgical tool with a circular cross-section diameter of 2 mm, can be imaged with less than 16 x-ray images.

For surgical navigation systems with higher accuracy limit (typically 1.5 mm), even fewer images may be sufficient for reconstruction. Further, for surgical navigation, the more important part of navigation is to track the tip of a surgical tool, which often has a relatively simple geometry and a small cross-sectional area, such as a sharp tip or a rectangle. Two images may be sufficient to reconstruct the cross-section of such a tool. Using geometrical information know for the object can reduce the number of images further. The reconstruction process may be performed iteratively using successive images to refine the reconstruction accuracy, e.g., until a threshold level of accuracy is determined. For example, successive images may be acquired and used to iteratively improve the digitized reconstructed shape until the improvement to the difference measurement, e.g., cost, (e.g., operation 206 in FIG. 2) using a last acquired image becomes less an minimum improvement threshold. In contrast, a determination that the last acquired image resulted in an improvement of more than the minimum improvement threshold can trigger another image of the object to be acquired at another pose.

Potential advantages that may be provided by one or more of the embodiments disclosed herein may include to enable overlay of an object CAD model on a 3D scan of a patient by taking fluoroscopy images or other 2D image modality (IES) showing the object implanted in the patient, and registering the images to the 3D scans. This allows the surgeon to avoid unknowingly performing a surgical procedure during computer-aided surgical navigation that results in getting too close or colliding with objects that are not captured in original 3D scans. The objects can include: radiographically opaque objects such as screws and interbodies, radiographically ‘dark’ contrast agents flowing through anatomy of known shape, such as cylindrical blood vessels, etc. The relative poses of the images may be determined based on information provided by the imaging devices or may be determined using other techniques, such as by camera tracking of tracking figures and possibly also a DRB attached to the patient. Improved accuracy of the object reconstruction can be provided by approximating the object CAD model to a known shape, such as a cylinder, so that a minimum of two fluoroscopy images are sufficient. Further accuracy may be provided when an exact CAD model of the object is identified, such as when the object is known to correspond to a CAD model, so that a minimum of two fluoroscopy images can be sufficient.

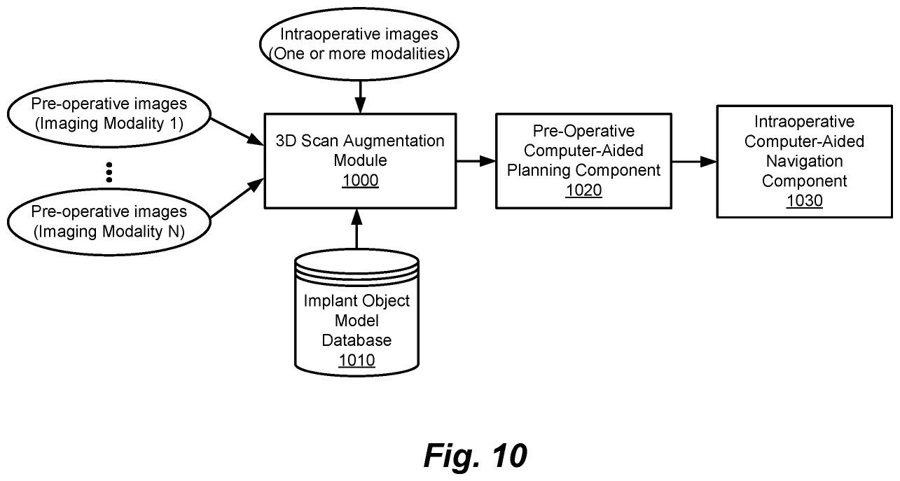

FIG. 10 illustrates another operational flowchart of an algorithm that augments a 3D scan of a patient to represent a CAD model corresponding to an object implanted in the patient, and which is used to perform pre-operative computer-aided planning and intraoperative computer-aided navigation in accordance with some embodiments.

Referring to FIG. 10, a 3D scan augmentation module 1000 obtains pre-operative images which may include one or more 3D imaging modalities, and obtains 2D images of an object implanted in a patient. The 2D images of the implanted object may be obtained as intraoperative images using one or more 2D imaging modalities. The 3D scan augmentation module 1000 also obtains a CAD model corresponding to the object. The 3D scan augmentation module 1000 may have access to a database 1010 of implantable objects, which defines for each object a CAD model that defines shape and physical dimensions. The 3D scan augmentation module 1000 augments a 3D scan of a patient to represent the CAD model corresponding to the object implanted in the patient, in accordance with one or more of the embodiments disclosed herein. The augmented 3D scan may be provided to a preoperative computer-aided planning component 1020 that enables a user (e.g., surgeon) to plan a surgical procedure for the patient, and generate therefore a computer readable surgical plan. The computer readable surgical plan and/or the augmented 3D scan may be provided to an intraoperative computer-aided navigation component that provides navigation information to a user (e.g., surgeon) to enable computer-aided guidance during the surgical procedure on the patient.

### Further Definitions and Embodiments

In the above-description of various embodiments of present inventive concepts, it is to be understood that the terminology used herein is for the purpose of describing particular embodiments only and is not intended to be limiting of present inventive concepts. Unless otherwise defined, all terms (including technical and scientific terms) used herein have the same meaning as commonly understood by one of ordinary skill in the art to which present inventive concepts belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

When an element is referred to as being “connected”, “coupled”, “responsive”, or variants thereof to another element, it can be directly connected, coupled, or responsive to the other element or intervening elements may be present. In contrast, when an element is referred to as being “directly connected”, “directly coupled”, “directly responsive”, or variants thereof to another element, there are no intervening elements present. Like numbers refer to like elements throughout. Furthermore, “coupled”, “connected”, “responsive”, or variants thereof as used herein may include wirelessly coupled, connected, or responsive. As used herein, the singular forms “a”, “an” and “the” are intended to include the plural forms as well, unless the context clearly indicates otherwise. Well-known functions or constructions may not be described in detail for brevity and/or clarity. The term “and/or” includes any and all combinations of one or more of the associated listed items.

It will be understood that although the terms first, second, third, etc. may be used herein to describe various elements/operations, these elements/operations should not be limited by these terms. These terms are only used to distinguish one element/operation from another element/operation. Thus, a first element/operation in some embodiments could be termed a second element/operation in other embodiments without departing from the teachings of present inventive concepts. The same reference numerals or the same reference designators denote the same or similar elements throughout the specification.

As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.”, which derives from the Latin phrase “exempli gratia,” may be used to introduce or specify a general example or examples of a previously mentioned item, and is not intended to be limiting of such item. The common abbreviation “i.e.”, which derives from the Latin phrase “id est,” may be used to specify a particular item from a more general recitation.

Example embodiments are described herein with reference to block diagrams and/or flowchart illustrations of computer-implemented methods, apparatus (systems and/or devices) and/or computer program products. It is understood that a block of the block diagrams and/or flowchart illustrations, and combinations of blocks in the block diagrams and/or flowchart illustrations, can be implemented by computer program instructions that are performed by one or more computer circuits. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart block or blocks, and thereby create means (functionality) and/or structure for implementing the functions/acts specified in the block diagrams and/or flowchart block(s).

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks. Accordingly, embodiments of present inventive concepts may be embodied in hardware and/or in software (including firmware, resident software, micro-code, etc.) that runs on a processor such as a digital signal processor, which may collectively be referred to as “circuitry,” “a module” or variants thereof.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Finally, other blocks may be added/inserted between the blocks that are illustrated, and/or blocks/operations may be omitted without departing from the scope of inventive concepts. Moreover, although some of the diagrams include arrows on communication paths to show a primary direction of communication, it is to be understood that communication may occur in the opposite direction to the depicted arrows.

Many variations and modifications can be made to the embodiments without substantially departing from the principles of the present inventive concepts. All such variations and modifications are intended to be included herein within the scope of present inventive concepts. Accordingly, the above disclosed subject matter is to be considered illustrative, and not restrictive, and the appended examples of embodiments are intended to cover all such modifications, enhancements, and other embodiments, which fall within the spirit and scope of present inventive concepts. Thus, to the maximum extent allowed by law, the scope of present inventive concepts are to be determined by the broadest permissible interpretation of the present disclosure including the following examples of embodiments and their equivalents, and shall not be restricted or limited by the foregoing detailed description.

1. A computer-implemented method comprising:
calculating an expected image of an object based on an object computer-aided design (CAD) model and estimated object poses;
comparing the expected image of the object to an actual image of the object obtained by an imaging device
calculating a difference measure between the expected image and the actual image as a cost; and
if the cost does not satisfy a rule, adjusting the estimated object poses, calculating a second expected image, and calculating a difference measure between the second expected image and the actual image until the cost meets the rule; or
if the cost does satisfy the rule, outputting the CAD model and estimated object poses.

2. The computer-implemented method of claim 1, wherein the object poses comprise one of more of position and angular orientation.

3. The computer-implemented method of claim 1, wherein the estimated object poses are determined based on tracked poses of an imaging device when actual images of the object are acquired.

4. The computer-implemented method of claim 1, wherein the cost does not satisfy the rule when it is above a pre-determined threshold.

5. The computer-implemented method of claim 1, wherein the cost does satisfy the rule when it is below a pre-determined threshold.

6. The computer-implemented method of claim 1, wherein outputting the CAD model and estimated object poses comprises overlaying the CAD model of the object in one or more of a two-dimensional image or a three-dimensional scan of a patient.

7. The computer-implemented method of claim 6, comprising overlaying the CAD model of the object in a two-dimensional image and estimating a pose of the object in the two-dimensional image.

8. The computer-implemented method of claim 7, comprising calculating expected object images based on features having a shape defined by the CAD model overlayed on the two-dimensional image based on the estimated pose of the object.

9. The computer-implemented method of claim 8, comprising comparing the expected images of the object to the CAD model overlayed on the two-dimensional image and calculating a difference measure, and determining whether the difference measure satisfies a second rule.

10. The computer-implemented method of claim 9, wherein, when the difference measure satisfies the second rule, determining a pose of the CAD model in a three-dimensional space based on the estimated pose of the object in the two-dimensional image.

11. The computer-implemented method of claim 10, comprising determining a pose of the CAD model in a reference system of a three-dimensional scan based on the pose of the CAD model in the three-dimensional space.
